(19) United States
(12) Patent Application Publication
(10) Pub. No.: US 2015/0013685 A1
(43) Pub. Date: Jan. 15, 2015

(54) PATIENT INTERFACE WITH EXPANDABLE NASAL PRONGS

(71) Applicant: Richard Thomas Haibach, Verona, PA (US)

(72) Inventor: Richard Thomas Haibach, Verona, PA (US)

(73) Assignee: KONINKLIJKE PHILIPS N.V., EINDHOVEN (NL)

(21) Appl. No.: 14/345,433

(22) PCT Filed: Aug. 31, 2012
(86) PCT No.: PCT/IB2012/054485

§ 371 Date: Mar. 18, 2014

Related U.S. Application Data
(60) Provisional application No. 61/537,848, filed on Sep. 22, 2011.

(51) Int. Cl. A61M 16/06

(52) U.S. Cl. CPC A61M 16/0666; A61M 16/0622; A61M 2205/0216; A61M 2210/0618
USPC 128/207.18

(57) ABSTRACT

A nasal cushion (102) for a patient interface device is provided that includes a main body portion (106) and a nasal prong (108A, 108B) extending from a top side of the main body portion. The nasal prong includes a base portion (112A, 112B) coupled to the main body portion and a top portion (114A, 114B) coupled to the base portion. A distal end of the top portion defines an expandable orifice structured (118A, 118B) to be switchable between an unexpanded state having a first diameter (dl) to an expanded state having a second diameter (d2) greater than the first diameter so as to reduce the jetting effect experienced by the patient.

This patent application claims the priority benefit under 35 U.S.C. §119(e) of U.S. Provisional Application No. 61/537,848 filed on Sep. 22, 2011, the contents of which are herein incorporated by reference.

### BACKGROUND OF THE INVENTION

1. Field of the Invention

The present invention relates to patient interface devices for transporting a gas to and/or from an airway of a user, and, in particular, to a patient interface device including a pillows style nasal cushion having expandable nasal prongs which reduce jetting within the nares of the patient.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion that rests beneath the patient's nose (such as a “pillows” style nasal cushion having nasal prongs that are received within the patient's nares or a “cradle” style nasal cushion that rests beneath and covers the patient's nares), a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

FIGS. 1 and 2 are schematic diagrams showing a prior art pillows style nasal cushion 2 inserted into the nose 4 (shown in cross-section) of a patient. FIG. 2 shows a side view of the entire nasal passage 5 of the patient. Nasal cushion 2 is made of a flexible, cushiony, elastomeric material, such as, without limitation, silicone, an appropriately soft thermoplastic elastomer, a closed cell foam, or any combination of such materials, and includes a main body portion 6 having nasal prongs 8A and 8B extending from a top side thereof and an orifice 10 structured to be fluidly coupled to a fluid coupling conduit (not shown), such as an elbow connector, that is coupled to a ventilator or pressure support device. Each nasal prong 8A, 8B is structured to be received within a respective nare 12A, 12B of the patient and has an orifice 14A, 14B having a fixed diameter (d) through which the flow of breathing gas is delivered from inside nasal cushion 2 to the nares 12A, 12B of the patient.

A common problem with prior art pillows style nasal cushion 2 is the unpleasant jetting effect that occurs within the patient's nares 12A, 12B. This jetting effect is due to the relatively small size of the orifices 14A, 14B, which causes a high velocity current of air (shown by the arrows in FIGS. 1 and 2) as a large volume of air passes from inside main body 6 through the small orifices 14A, 14B. This effect can be reduced by increasing the diameter of the orifices 14A, 14B, thus decreasing the velocity. Simply increasing the diameter, however, makes is difficult to lead nasal prongs 8A, 8B into nares 12A, 12B.

It can thus be appreciated that the present invention provides a nasal pillows style patient interface device having a mechanism for automatically reducing the jetting effect to improve patient comfort.

### SUMMARY OF THE INVENTION

Accordingly, it is an object of the present invention to provide a patient interface device that overcomes the shortcomings of conventional patient interface devices. This object is achieved according to one embodiment of the present invention by providing a patient interface device that includes a pillows style nasal cushion that automatically increases the diameter of the nasal prongs to automatically reduce the jetting effect of the flow of breathing gas into the patients nares.

In one embodiment, a nasal cushion for a patient interface device is provided that includes a main body portion and a nasal prong extending from a top side of the main body portion, the nasal prong including a base portion coupled to the main body portion and a top portion coupled to the base portion. A distal end of the top portion defines an expandable orifice structured to be switchable between an unexpanded state having a first diameter to an expanded state having a second diameter greater than the first diameter. The top portion includes a lever member extending outwardly from a middle portion of the top portion, wherein movement of the lever member in a downward direction toward the main body portion (as a result of being engaged and pushed by the patient's nares) causes the expandable orifice to move from the unexpanded state to the expanded state.

In another embodiment, a nasal cushion for a patient interface device is provided that includes a main body portion and a nasal prong extending from a top side of the main body portion, the nasal prong including a base portion coupled to the main body portion and a top portion coupled to the base portion. A distal end of the top portion defines an expandable orifice structured to be switchable between an unexpanded state having a first diameter to an expanded state having a second diameter greater than the first diameter. The top portion includes a plurality of inwardly extending pleat members provided around a periphery of the top portion, wherein the pleat members are structured to be in a closed condition responsive to no gas pressure being present within the nasal cushion such that the expandable orifice is in the unexpanded state and to move to an open condition responsive to gas pressure above a certain level being present within the nasal cushion such that the expandable orifice is in the expanded state.

In yet another embodiment, a nasal cushion for a patient interface device is provided that includes a main body portion and a nasal prong extending from a top side of the main body portion, the nasal prong including a base portion coupled to the main body portion and a top portion coupled to the base portion. A distal end of the top portion defines an expandable orifice structured to be switchable between an unexpanded state having a first diameter to an expanded state having a second diameter greater than the first diameter. The top portion includes a plurality of slits therein provided around a periphery of the top portion, each of the slits extending from a middle portion of the top portion to the distal end of the top portion, wherein the slits are structured to be in a closed condition responsive to no gas pressure being present within the nasal cushion such that the expandable orifice is in the unexpanded state and to separate responsive to gas pressure above a certain level being present within the nasal cushion such that the expandable orifice is in the expanded state.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIGS. 1 and 2 are schematic diagrams showing a prior art pillows style nasal cushion inserted into the nose of a patient;

FIG. 2 is a schematic diagram of a system adapted to provide a regimen of respiratory therapy to a patient according to one exemplary embodiment of the present invention;

FIGS. 3 and 4 are front elevational views of a nasal cushion forming a part of the system of FIG. 2 according to an exemplary embodiment of the present invention shown in both an unexpanded state (FIG. 3) and an expanded state (FIG. 4);

FIGS. 5A and 5B are schematic diagrams illustrating the operation of the nasal cushion of FIGS. 3 and 4;

FIGS. 6 and 7 are front elevational views of a nasal cushion according to an alternative exemplary embodiment of the present invention that may form a part of the system of FIG. 2 shown in both an unexpanded state (FIG. 6) and an expanded state (FIG. 7);

FIGS. 8 and 9 are front elevational views of a nasal cushion according to a further alternative exemplary embodiment of the present invention that may form a part of the system of FIG. 2 shown in both an unexpanded state (FIG. 6) and an expanded state (FIG. 7);

FIGS. 10 and 11 are cross-sectional views of a nasal prong of the nasal cushion of FIGS. 8 and 9;

FIG. 12 is a cross-sectional view of an alternative embodiment of a nasal prong that may form part of the nasal cushion of FIGS. 3 and 4;

FIG. 13 is a cross-sectional view of an alternative embodiment of a nasal prong that may form part of the nasal cushion of FIGS. 6 and 7; and

FIGS. 14A and 14B are partial cross-sectional views of the nasal prong of FIGS. 8 and 9 according to one particular exemplary embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

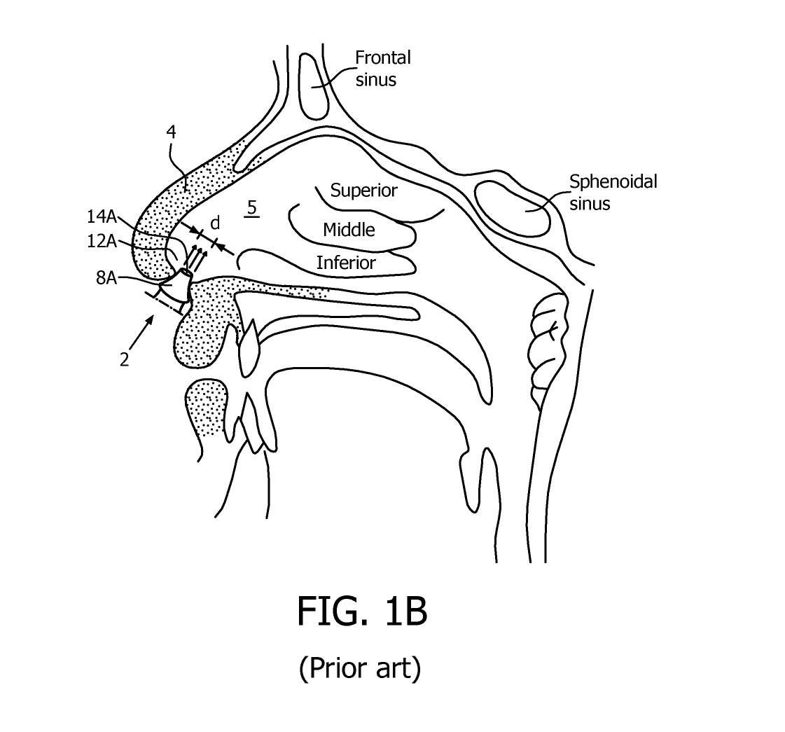

A system 20 adapted to provide a regimen of respiratory therapy to a patient according to one exemplary embodiment is generally shown in FIG. 2. System 20 includes a pressure generating device 22, a delivery conduit 24, and a patient interface device 26 having a fluid coupling conduit 28. Pressure generating device 22 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 24 is structured to communicate the flow of breathing gas from pressure generating device 22 to patient interface device 26 through fluid coupling conduit 28, which in the illustrated embodiment is an elbow connector. Delivery conduit 24 and patient interface device 26 are often collectively referred to as a patient circuit.

As seen in FIG. 2, patient interface 26 includes a patient sealing assembly 30 which facilitates the delivery of the flow of breathing gas to the airway of a patient. Patient sealing assembly 30 includes a frame member 32 having a cushion assembly 34 coupled thereto. In the illustrated embodiment, frame member 32 is made of a rigid or semi-rigid material, such as, without limitation, an injection molded thermoplastic or silicone. Frame member 32 includes a generally flat central portion 36 positioned beneath cushion assembly 34. Frame member 32 further includes arms 38A and 38B positioned on opposite sides of central portion 36. Each arm 38A, 38B includes a looped connector 40A, 40B structured to enable a headgear strap (not shown) to be attached to frame member 32 in a known manner.

Cushion assembly 34 includes a nasal cushion 42 structurally and fluidly coupled to the rear side of a sub-frame member 44. Sub-frame member 44 is made of a rigid or semi-rigid material, such as, without limitation, an injection molded thermoplastic or silicone. In addition, fluid coupling conduit 28 is coupled to the front side of sub-frame member 44. This configuration allows the flow of breathing gas from pressure generating device 22 to be communicated to nasal cushion 42, and then to the airway of a patient.

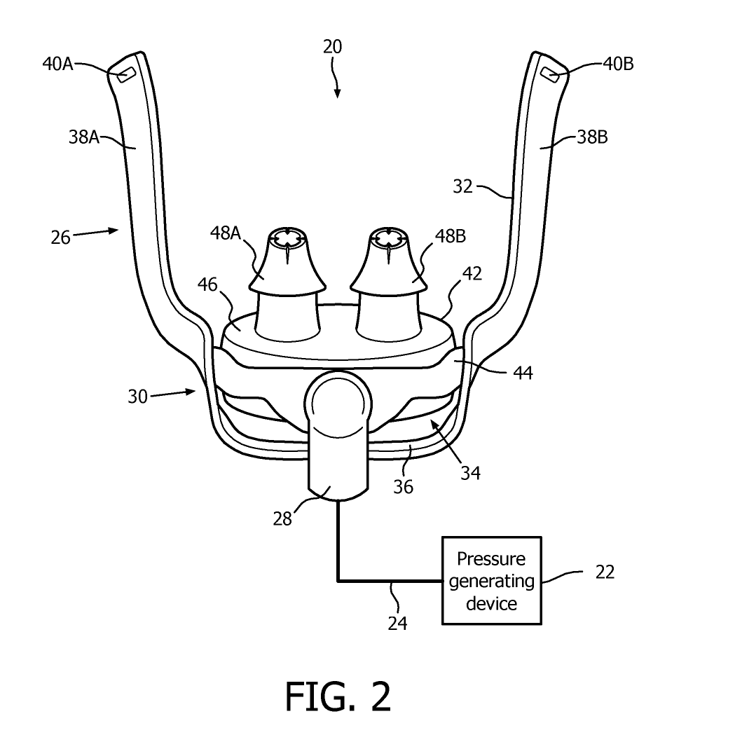

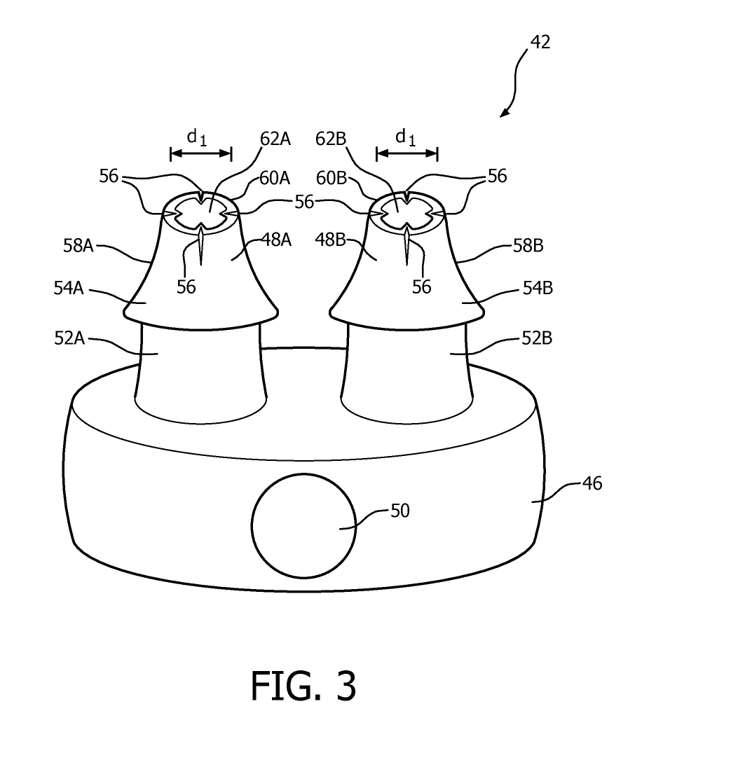

FIGS. 3 and 4 are front elevational views of nasal cushion 42 according to the exemplary embodiment of the present invention shown in both an unexpanded state (FIG. 3) and an expanded state (FIG. 4), each of which is described below. Nasal cushion 42 is a pillows style nasal cushion made of a flexible, cushiony, elastomeric material, such as, without limitation, silicone, an appropriately soft thermoplastic elastomer, a closed cell foam, or any combination of such materials. Nasal cushion 42 made be made using, for example, an injection or compression molding technique. Nasal cushion 42 includes a main body portion 46 having nasal prongs 48A and 48B extending from a top side thereof. Nasal cushion 42 also includes an orifice 50 structured to be fluidly coupled to fluid coupling conduit 28. Each nasal prong 48A, 48B is structured to be received within a respective nare of the patient and includes a base portion 52A, 52B coupled to main body portion 46 and a top portion 54A, 54B coupled to the base portion 52A, 52B. As seen in FIGS. 3 and 4, each top portion 54A, 54B has a plurality of inwardly extending pleat members 56 provided in an outer surface thereof. In the illustrated embodiment, pleat members 56 extend from a middle portion 58A, 58B of each top portion 54A, 54B to a terminal/distal end 60A, 60B of each top portion 54A, 54B.

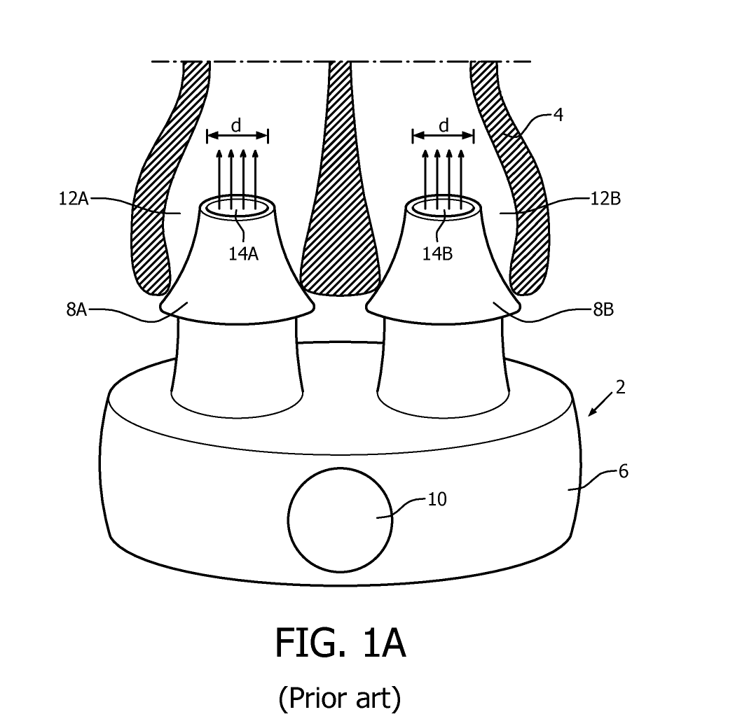

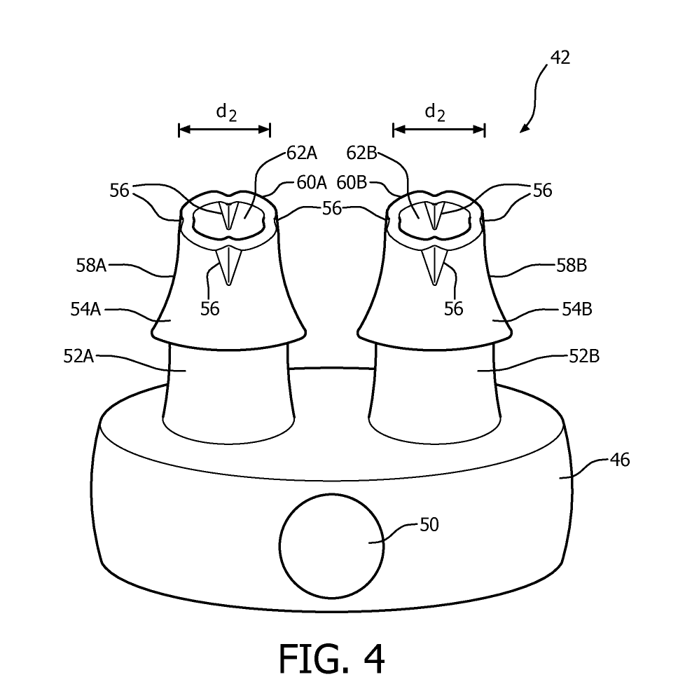

Furthermore, each terminal/distal end 60A, 60B defines an expandable orifice 62A, 62B that is structured to be switchable between an unexpanded state (FIG. 3) having a diameter d₁ to an expanded state (FIG. 4) having a diameter d₂, wherein d₂>d₁. In particular, nasal prongs 48A, 48B are structured such that, when no gas is being provided to nasal cushion 42 through orifice 50, pleat members 56 will remain in a closed (folded) condition and expandable orifices 62A, 62B will be in the unexpanded state of FIG. 3. In this state, the smaller diameter d₁ permits nasal prongs 48A and 48B to be easily inserted into the patient's nares. When a flow of breathing gas is subsequently provided to nasal cushion 42 through orifice 50, the gas pressure within nasal cushion 42 will cause pleat members 56 to automatically open/expand, thereby increasing the diameter of expandable orifices 62A, 62B to the expanded diameter d₂ shown in FIG. 4. In this expanded state, the cross-sectional area of orifices 62A, 62B, and thus the cross-sectional area of the air flow path, is maximized, which reduces the pressure drop from the interior of main body portion 46 and reduces the jetting effect (as compared to the prior art (FIG. 1)). This result is also illustrated in FIGS. 5A and 5B. Thus, nasal cushion 42 provides a mechanism wherein the therapy pressure delivered to the patient from pressure generating device 22 automatically increases the diameter of orifices 62A, 62B of nasal prongs 48A, 48B to reduce the jetting effect experienced by the patient.

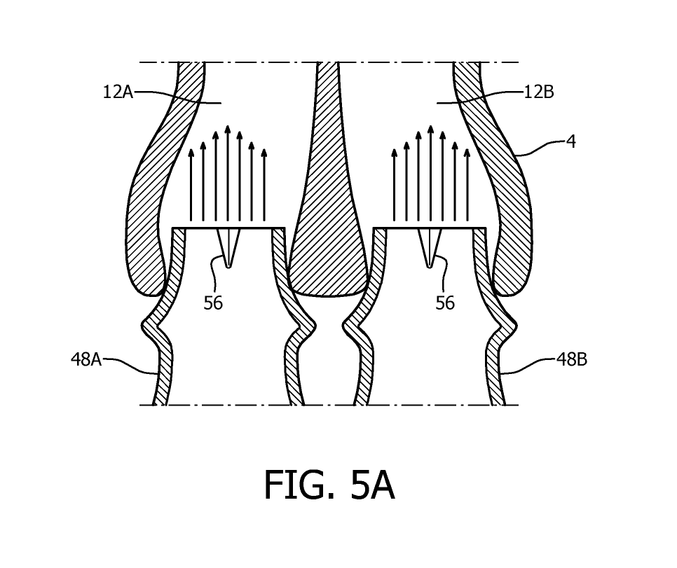

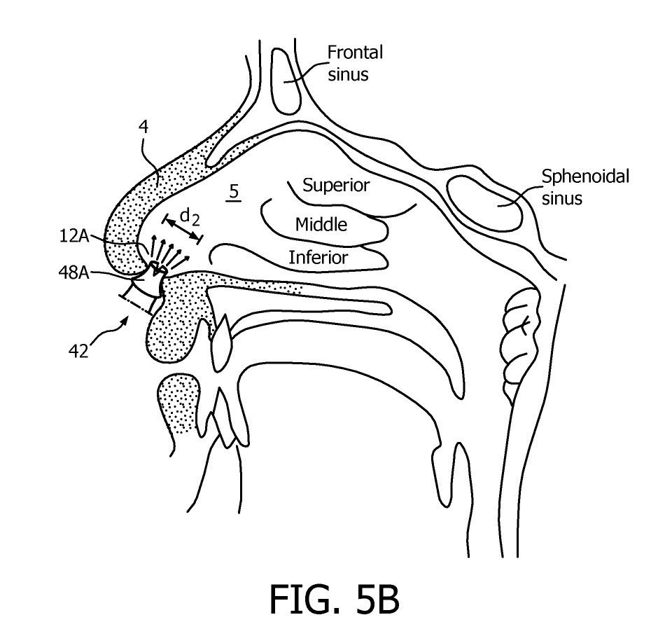

FIGS. 6 and 7 are front elevational views of a nasal cushion 72 according to an alternative exemplary embodiment of the present invention shown in both an unexpanded state (FIG. 6) and an expanded state (FIG. 7), each of which is described below. Nasal cushion 72 may be substituted for nasal cushion 42 in patient interface device 26 of system 20. Nasal cushion 72 is also a pillows style nasal cushion made of a flexible, cushiony, elastomeric material, such as, without limitation, silicone, an appropriately soft thermoplastic elastomer, a closed cell foam, or any combination of such materials. Nasal cushion 72 includes a main body portion 76 having nasal prongs 78A and 78B extending from a top side thereof. Nasal cushion 72 also includes an orifice 80 structured to be fluidly coupled to fluid coupling conduit 28. Each nasal prong 78A, 78B is structured to be received within a respective nare of the patient and includes a base portion 82A, 82B coupled to main body portion 76 and a top portion 84A, 84B coupled to the base portion 82A, 82B. As seen in FIGS. 6 and 7, each top portion 84A, 84B has a plurality of slits 86 provided in an outer surface thereof. In the illustrated embodiment, slits 86 extend from a middle portion 88A, 88B of each top portion 84A, 84B to a terminal/distal end 90A, 90B of each top portion 84A, 84B.

Furthermore, each terminal/distal end 90A, 90B defines an expandable orifice 92A, 92B that is structured to be switchable between an unexpanded state (FIG. 6) having a diameter d₁ to an expanded state (FIG. 7) having a diameter d₂, wherein d₂>d₁. In particular, nasal prongs 78A, 78B are structured such that, when no gas is being provided to nasal cushion 72 through orifice 80, slits 86 will remain in a closed condition and expandable orifices 92A, 92B will be in the unexpanded state of FIG. 6. In this state, the smaller diameter d₁ permits nasal prongs 78A and 78B to be easily inserted into the patient's nares. When a flow of breathing gas is subsequently provided to nasal cushion 72 through orifice 80, the gas pressure within nasal cushion 72 will cause slits 86 to automatically open/separate, thereby increasing the diameter of expandable orifices 92A, 92B to the expanded diameter d₂ shown in FIG. 7. In this expanded state, the cross-sectional area of orifices 92A, 92B, and thus the cross-sectional area of the air flow path, is maximized, which reduces the pressure drop from the interior of main body portion 76 and reduces the jetting effect (as compared to the prior art (FIG. 1)). A result similar to that illustrated in FIGS. 5A and 5B is provided. Thus, nasal cushion 72, like nasal cushion 42, provides a mechanism wherein the therapy pressure delivered to the patient from pressure generating device 22 automatically increases the diameter of orifices 92A, 92B of nasal prongs 78A, 78B to reduce the jetting effect experienced by the patient.

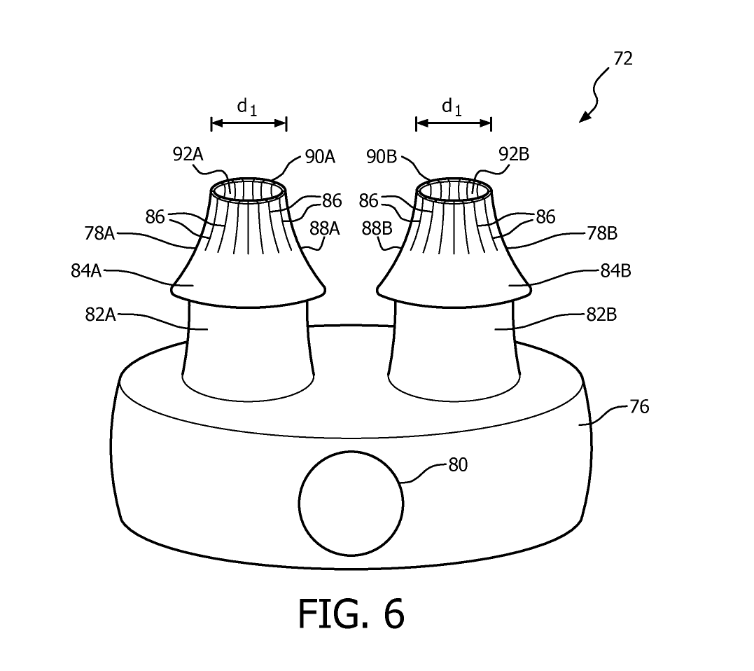

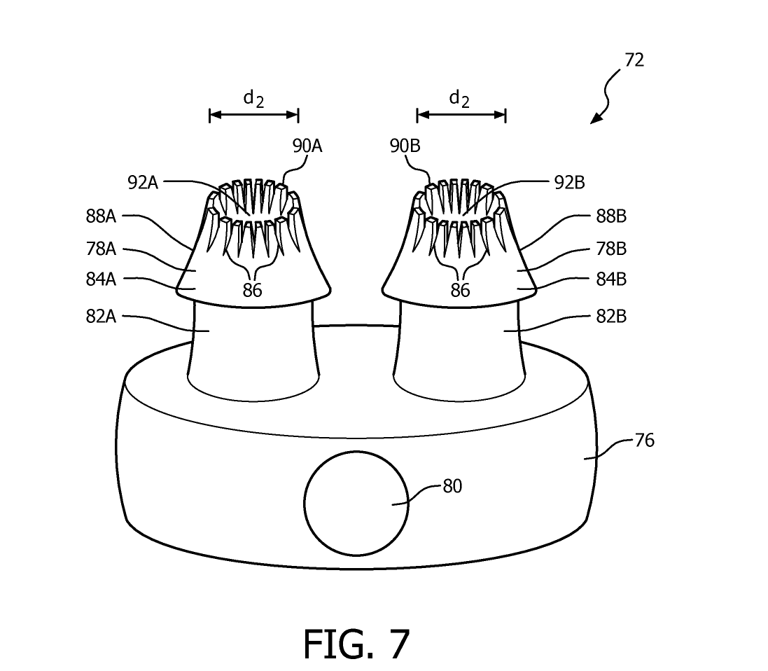

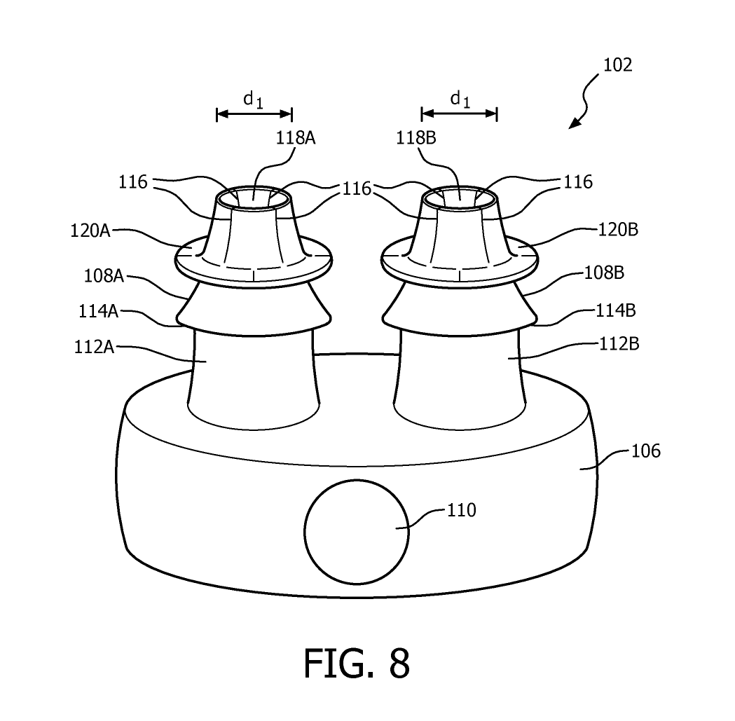

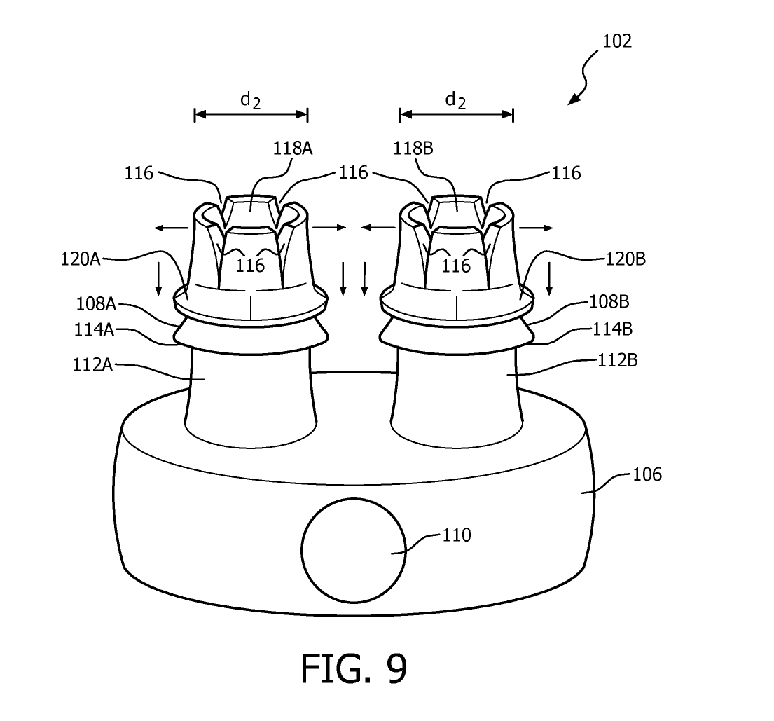

FIGS. 8 and 9 are front elevational views of a nasal cushion 102 according to a further alternative exemplary embodiment of the present invention shown in both an unexpanded state (FIG. 8) and an expanded state (FIG. 9), each of which is described below. Nasal cushion 102 may be substituted for nasal cushion 42 in patient interface device 26 of system 20. Nasal cushion 102, like nasal cushions 42 and 72, is a pillows style nasal cushion made of a flexible, cushiony, elastomeric material, such as, without limitation, silicone, an appropriately soft thermoplastic elastomer, a closed cell foam, or any combination of such materials. Nasal cushion 102 includes a main body portion 106 having nasal prongs 108A and 108B extending from a top side thereof. Nasal cushion 102 also includes an orifice 110 structured to be fluidly coupled to fluid coupling conduit 28. FIGS. 10 and 11 are cross-sectional views of one of the nasal prongs 108A, 108B illustrating both the unexpanded state (FIG. 10) and the expanded state (FIG. 11), described in greater detail below.

Each nasal prong 108A, 108B is structured to be received within a respective nare of the patient and includes a base portion 112A, 112B coupled to main body portion 106 and a top portion 114A, 114B coupled to the base portion 112A, 112B. As seen in FIGS. 8-11, each top portion 114A, 114B has a plurality of slits 116 provided in an outer surface thereof. In the illustrated embodiment, slits 116 extend from a middle portion of each top portion 114A, 114B to a terminal/distal end of each top portion 114A, 114B. In an alternative embodiment, slits 116 may be replaced by pleats such as pleats 56 described elsewhere herein. Furthermore, each terminal/distal end of each top portion 114A, 114B defines an expandable orifice 118A, 118B that is structured to be switchable between an unexpanded state (FIGS. 8 and 10) having a diameter d₁ to an expanded state (FIGS. 9 and 11) having a diameter d₂, wherein d₂>d₁.

In addition, each top portion 114A, 114B includes a circular lever member 120A, 120B extending outwardly from the middle portion thereof. Each circular lever member 120A, 120B functions as a mechanical opening mechanism for moving the associated nasal prong 108A, 108B from the unexpanded state to the expanded state. In particular, nasal prongs 108A, 108B are structured such that, when no downward force is being applied to circular lever members 120A, 120B by the nares of the patient (FIG. 10), slits 116 will remain in a closed condition and expandable orifices 118A, 118B will be in the unexpanded state. In this state, the smaller diameter d₁ permits nasal prongs 108A and 108B to be easily inserted into the patient's nares. However, when nasal prongs 108A and 108B are inserted into the patient's nares, the patient's nares will engage the circular lever members 120A, 120B and will exert a downward force on the circular lever members 120A, 120B (as indicated by the downward facing arrows in FIG. 11). In response, the circular lever members 120A, 120B will be moved downwardly, which will in turn cause slits 116 to automatically open/separate, thereby increasing the diameter of expandable orifices 118A, 118B to the expanded diameter d₂ shown in FIG. 11. In this expanded state, the cross-sectional area of orifices 118A, 118B, and thus the cross-sectional area of the air flow path, is maximized, which reduces the pressure drop from the interior of main body portion 106 and reduces the jetting effect (as compared to the prior art (FIG. 1)). A result similar to that illustrated in FIGS. 5A and 5B is provided. Thus, nasal cushion 102 provides a patient initiated mechanical mechanism that automatically, during normal use, increases the diameter of orifices 118A, 118B of nasal prongs 108A, 108B to reduce the jetting effect experienced by the patient.

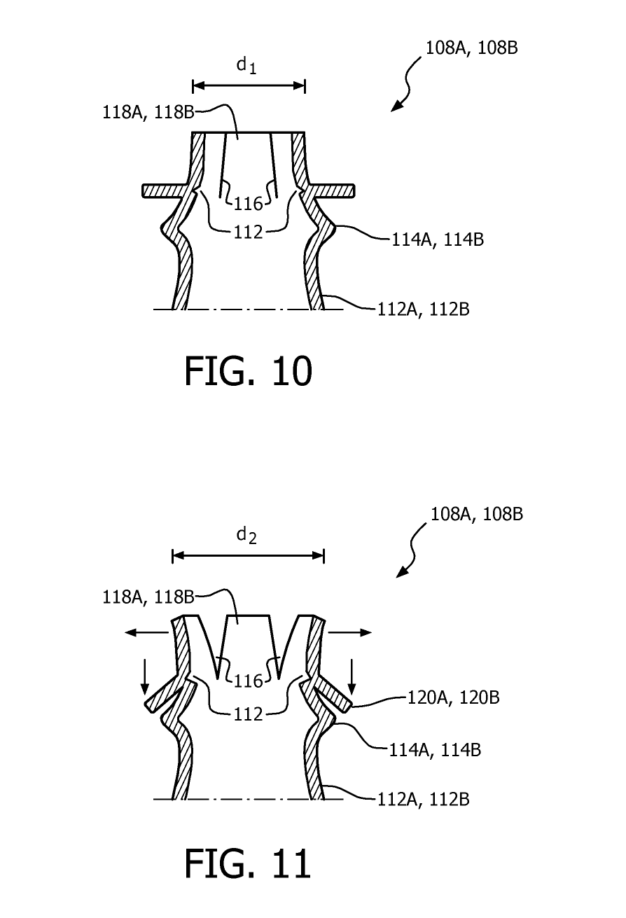

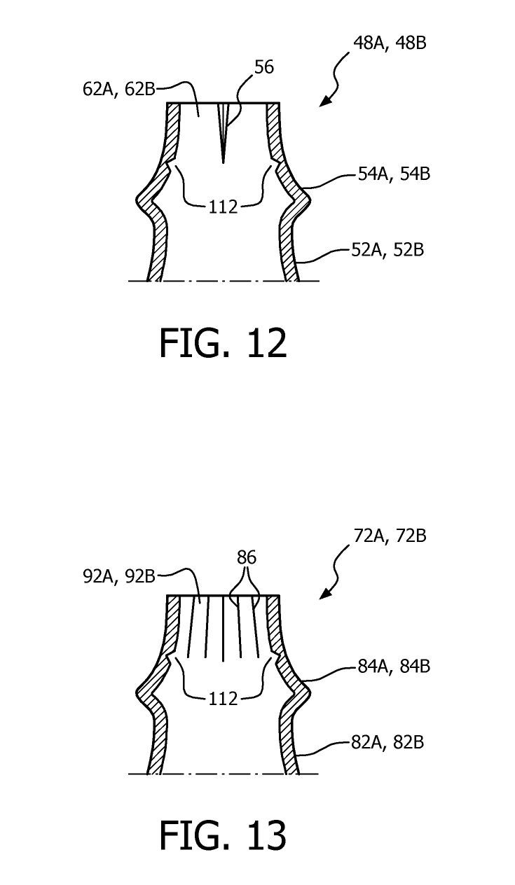

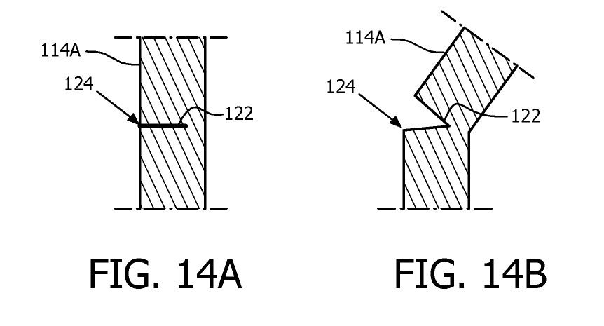

Moreover, in the illustrated embodiment shown in FIGS. 10 and 11, a hinge member 122 is integrally molded into the middle portion of each top portion 114A, 114B (at a base position just below the bottom of the slits 116 and adjacent the circular members 120A, 120B). The hinge members 122 facilitate the expansion of the orifices 118A, 118B. In addition, the hinge members 122 may be designed to move outwardly more easily than inwardly, thereby increasing the ease of expanding the orifices 118A, 118B. For example, each hinge members 122 may have a stop mechanism 124, restricting it from moving inward as shown in, for example, FIGS. 14A and 14B. In addition, as shown in FIGS. 12 and 13, hinge member 122 may also be provided in nasal prongs 48A, 48B and/or nasal prongs 78A, 78B to form alternative embodiments thereof.

It can thus be appreciated that the present invention provides a nasal pillows style patient interface device having a mechanism for automatically reducing the jetting effect to improve patient comfort.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A nasal cushion for a patient interface device, comprising:
a main body portion; and
a nasal prong extending from a top side of the main body portion, the nasal prong including a base portion coupled to the main body portion and a top portion coupled to the base portion, a bottom proximal edge of the top portion extending radially outwardly from the base portion, a distal end of the top portion defining an expandable orifice structured to be switchable between an unexpanded state having a first diameter to an expanded state having a second diameter greater than the first diameter, wherein the top portion includes a lever member provided in between the bottom proximal edge and the distal end and extending outwardly from a middle portion of the top portion, wherein movement of the lever member in a downward direction toward the main body portion causes the expandable orifice to move from the unexpanded state to the expanded state.

2. The nasal cushion according to claim 1, wherein the lever member has a circular shape and extends around an entire outer periphery of the top portion.

3. The nasal cushion according to claim 1, wherein the top portion includes a plurality of slits provided therein.

4. The nasal cushion according to claim 3, wherein each of the slits extends from the middle portion to the distal end of the top portion.

5. The nasal cushion according to claim 1, wherein the top portion includes a plurality of pleats provided therein.

6. The nasal cushion according to claim 5, wherein each of the pleats extends from the middle portion to the distal end of the top portion.

7. The nasal cushion according to claim 1, wherein the top portion further includes a hinge member provided on an inner surface thereof.

8. The nasal cushion according to claim 7, wherein the hinge member extends around an entire inner periphery of the top portion.

9. A nasal cushion for a patient interface device, comprising:
a main body portion; and
a nasal prong extending from a top side of the main body portion, the nasal prong including a base portion coupled to the main body portion and a top portion coupled to the base portion, a distal end of the top portion defining an expandable orifice structured to be switchable between an unexpanded state having a first diameter to an expanded state having a second diameter greater than the first diameter, wherein the top portion includes a plurality of inwardly extending pleat members provided around a periphery of the top portion, wherein the pleat members are structured to be in a closed condition responsive to no gas pressure being present within the nasal cushion such that the expandable orifice is in the unexpanded state and to move to an open condition responsive to gas pressure above a certain level being present within the nasal cushion such that the expandable orifice is in the expanded state.

10. The nasal cushion according to claim 9, wherein each of the pleats extends from a middle portion of the top portion to the distal end of the top portion.

11. The nasal cushion according to claim 9, wherein the top portion further includes a hinge member provided on an inner surface thereof below a bottom of each of the pleats.

12. The nasal cushion according to claim 11, wherein the hinge member extends around an entire inner periphery of the top portion.

13. A nasal cushion for a patient interface device, comprising:
a main body portion; and
a nasal prong extending from a top side of the main body portion, the nasal prong including a base portion coupled to the main body portion and a top portion coupled to the base portion, a distal end of the top portion defining an expandable orifice structured to be switchable between an unexpanded state having a first diameter to an expanded state having a second diameter greater than the first diameter, wherein the top portion includes a plurality of slits therein provided around a periphery of the top portion, each of the slits extending from a middle portion of the top portion to the distal end of the top portion, wherein the slits are structured to be in a closed condition responsive to no gas pressure being present within the nasal cushion such that the expandable orifice is in the unexpanded state and to separate responsive to gas pressure above a certain level being present within the nasal cushion such that the expandable orifice is in the expanded state.

14. The nasal cushion according to claim 13, wherein the top portion further includes a hinge member provided on an inner surface thereof below a bottom of each of the slits.

15. The nasal cushion according to claim 14, wherein the hinge member extends around an entire inner periphery of the top portion.
